(19) United States
(12) Patent Application Publication
(10) Pub. No.: US 2006/0264761 A1
(43) Pub. Date: Nov. 23, 2006

(54) Portable fluorescence scanner for molecular signatures

(76) Inventors: Jochem Knoche, Erlangen (DE); Wolfgang Strob, Erlangen (DE)

Correspondence Address: BRINKS HOFER GILSON & LIONE, P.O. BOX 10395, CHICAGO, IL, 60610, US

(21) Appl. No.: 11/375,934

(22) Filed: Mar. 15, 2006

(30) Foreign Application Priority Data
Mar. 18, 2005 (DE) 10 2005 013 043.7

(51) Int. Cl. A61B 6/00

(52) U.S. Cl. 600/476

(57) ABSTRACT

A fluorescence scanner, having a light source for generating excitation light, a detector for detecting fluorescent light, and a data acquisition unit. The excitation light source is operated in pulsed fashion. The pulsed mode results in short exposure times for the fluorescence images, so that artifacts caused by motion are reduced.

### RELATED APPLICATIONS

This application claims the benefit of German Patent application DE 10 2005 013 43.7, filed on Mar. 18, 2005, which is incorporated herein by reference.

The application relates to a device for detecting fluorescence.

### BACKGROUND

Equipment for fluorescence detection, hereinafter also called fluorescence scanners, can be used to detect various molecular factors. Substances having different molecular properties can have different fluorescent properties, which can be detected in a targeted way. The fluorescence detection is optically based and hence is noninvasive or only minimally invasive. With the knowledge of the applicable fluorescent properties, it is possible to ascertain the molecular nature of a given material being examined.

In medicine, molecular properties, which may be termed a “molecular signature”, provide information about the state of health of a living creature or patient and can therefore be assessed diagnostically. Molecular signatures can be used in particular for detecting cancer. Still other syndromes, such as rheumatoid arthritis or arteriosclerosis of the carotid artery, can thus be identified.

Fluorescence may be excited by optical excitation. The excitation light can be in the infrared range (IR), for example, or in the near infrared range (NIR). The suitable optical frequency range is also dependent on the substance to be examined. Substances that themselves have no molecular or chemical properties that would be suitable for fluorescence detection can be molecularly “marked”. For example, markers that with suitable preparation to bind to or to be deposited only on very special molecules can be used. Such marking functions by a mechanism that in pictorial terms can be thought of as a lock-and-key mechanism. The marker and the molecule to be detected fit one another like a lock and key, while the marker does not bind to other substances. If the marker has known fluorescent properties then, after the binding or deposition, the marker can be optically detected. The detection of the marker then allows conclusions to be drawn as to the presence of the marked special substance. For detection, only one detector is needed. The detector is capable of detecting light in the wavelength of the substance in question, or the marker used upon excitation.

Such fluorescence methods may be used for examinations of regions near the surface or examinations in the open body (intra-operative applications). Examples of such investigations would be detecting fluorescently marked skin cancer or the detection of tumor boundaries in the resection of fluorescently marked tumors. For example, a system for making coronary arteries and the function of bypasses (that is, the blood flow through them) visible intra-operatively has been developed.

One subject of research in biotechnology is fluorescent metabolic markers that accumulate only in certain regions (such as tumors, infections, or other foci of disease), or are distributed throughout the body but are activated only in certain regions. Activation may be by tumor-specific enzyme activities or, for example, by additional exposure to light.

In medical diagnosis, marker substances, so-called fluorophores such as indocyanin green (ICG) are known, which for example bind to blood vessels and can be detected optically, so that in an imaging process, the contrast with which blood vessels are displayed can be enhanced. So-called “smart contrast agents” are also becoming increasingly important. Activatable fluorescence markers that may bind, for example, to tumor tissue and the fluorescent properties are not activated until the binding to the substance to be marked occurs. Such substances may comprise self-quenched dyes, such as Cy5.5, which are bound to larger molecules by way of specific peptides. The peptides can in turn be detected by means of specific proteases, produced for example in tumors, and can be cleaved. The fluorophores are released by the cleavage and are no longer self-quenched but instead develop their fluorescent properties. The released fluorophores can be activated for example in the near IR wavelength range of around 740 nm. One example of a marker on this basis is AF 750 (Alexa Fluor 750), with a defined absorption and emission spectrum in the wavelength range of 750 nm (excitation) and 780 nm (emission).

In medical diagnosis, such activatable markers can be used for example for intra-operative detection of tumor tissue, so that the diseased tissue can be identified exactly and then removed. One typical application is the surgical treatment of ovarian cancer. Here, the diseased tissue is typically removed surgically, and the patient later treated by chemotherapy. Because of the increased sensitivity of fluorescence detection, the diseased tissue can be better detected along with various surrounding foci of disease and thus removed more completely.

In the treatment of breast cancer, typical surgical treatments are lumpectomies (or mastectomies) and lymph node sections and lymph node biopsies. Lymph nodes are typically detected optically by means of 99mTc sulfur colloids in combination with low-molecular methylene blue. The radioactive mTc sulfur colloids could be avoided by using fluorescence detection, with correspondingly favorable effects on the health of the patient.

In the removal of brain tumors, the precise demarcation of the tumor tissue, which is attainable by the use of fluorescence detection, is of obvious importance. The treatment of pancreatic tumors can benefit from additional lymph node biopsies which could be identified by fluorescence detection, to detect possible intestinal cancer. In the treatment of skin cancer, the detection of skin neoplasms could be improved by fluorescence detection. The treatment of rheumatoid arthritic diseases of joints could improve medication monitoring in the sense that the extent of protease overproduction could be detected quantitatively, and the medication provided to counteract protease overproduction could be adapted quantitatively.

In treating these diseases which are identified as examples, as well as other syndromes, an operation may be performed in which the diseased tissue is removed surgically. Fluorescence detection can be performed to improve the detection of the diseased tissue portions to be removed during an ongoing operation, in the open wound. The tissue parts must be marked before the operation with a suitable substance that is then activated by binding to the diseased tissue parts. An apparatus for fluorescence detection should therefore be easy for the surgeon to manipulate and should be usable in the sterile field of the operating room.

The detection of a region marked fluorescently in this way is done by exposing the region to light in the particular excitation wavelength of the fluorescent dye, and detecting the emitted light in the corresponding emission wavelength of the fluorophore. A fluorescence scan is made by recording a fluorescence image based on fluorescent light along with an optical image based on visible light. Next, the optical image and the fluorescence image are superimposed, in order to display the fluorescence in the context of the visual image. From the superimposed view (fusion) of the optical and fluorescence images on a display device, the surgeon can detect the tumor tissue and locate it in the body of the actual patient. The fused image with the fluorescently marked tissue is displayed on a screen on the fluorescence scanner or on an external computer with image processing software.

Typically, the excitation of the fluorescence of the marker is done by means of light, and the detection device must have a light source of adequate intensity, in order to penetrate the tissue to be examined to a depth of from 0.5 to 1 cm. In addition, an optical detector is necessary that on the one hand is capable of detecting the fluorescent light and on the other, if the fluorescent light is not in the visible wavelength range, also to record an image in the visible wavelength range.

The fluorescent light in question is often in the infrared wavelength range (IR) or the near infrared wavelength range (NIR). Excitation light of a suitable wavelength, which for fluorescence is typically in the near IR range up to 700 nm, and adequate intensity for sufficient penetration of tissue can be attained with the known illuminants only with relatively low efficiency. Given adequate intensity in the wavelength range of interest, the heat production is enormous, because of the low efficiency. Simultaneously, the energy consumption for generating the excitation light is considerable. A power-cord energy supply for furnishing the required energy would make the device inconvenient to manipulate in the operating room area, where work must be done in a restricted space. Moreover, in the sterile field, active cooling of the illuminants, for example by fans, cannot be done since adequate sterilization of an actively cooled device is difficult.

### SUMMARY

The device includes an energy source, at least one light source for generating excitation light, at least one detector for detecting fluorescent light, and a data acquisition unit. The excitation light source operates in a pulsed fashion. The pulsed mode reduces both energy consumption and the concomitant heat produced. A power supply cable and active cooling may be avoided. Furthermore, the pulsed mode results in short exposure times for the images, so that artifacts caused by motion are reduced. Configurations of the fluorescence sensor may be portable and sterilizable. The image detector may for example be a CCD camera, but other picture-taking technologies can be used.

### BRIEF DESCRIPTION OF THE DRAWINGS

FIG. 1 is an application scenario for a fluorescence scanner according to one embodiment;

FIG. 2 is a perspective view of an embodiment of a fluorescence scanner with the top cover removed;

FIG. 3 is a side view of one embodiment of a fluorescence scanner;

FIG. 4 is a time history graph of the actuating pulse and the operating pulse of the excitation light source in one embodiment; and

FIG. 5 is a time history graph of the actuating pulse and a train of operating pulses of the excitation light source according to one embodiment.

### DETAILED DESCRIPTION OF THE DRAWINGS AND THE PRESENTLY PREFERRED EMBODIMENTS

Exemplary embodiments may be better understood with reference to the drawings, but these embodiments are not intended to be of a limiting nature. Like numbered elements in the same or different drawings perform equivalent functions.

A fluorescence scanner, having at least one light source for generating excitation light, at least one detector for detecting fluorescent light, and a data acquisition unit is described. The excitation light source is operated in pulsed fashion, possibly reducing energy consumption and heat dissipation. The pulsed mode may result in short exposure times for the fluorescence images, so that image artifacts caused by motion are reduced. A small, portable device that may be sterilizable may result.

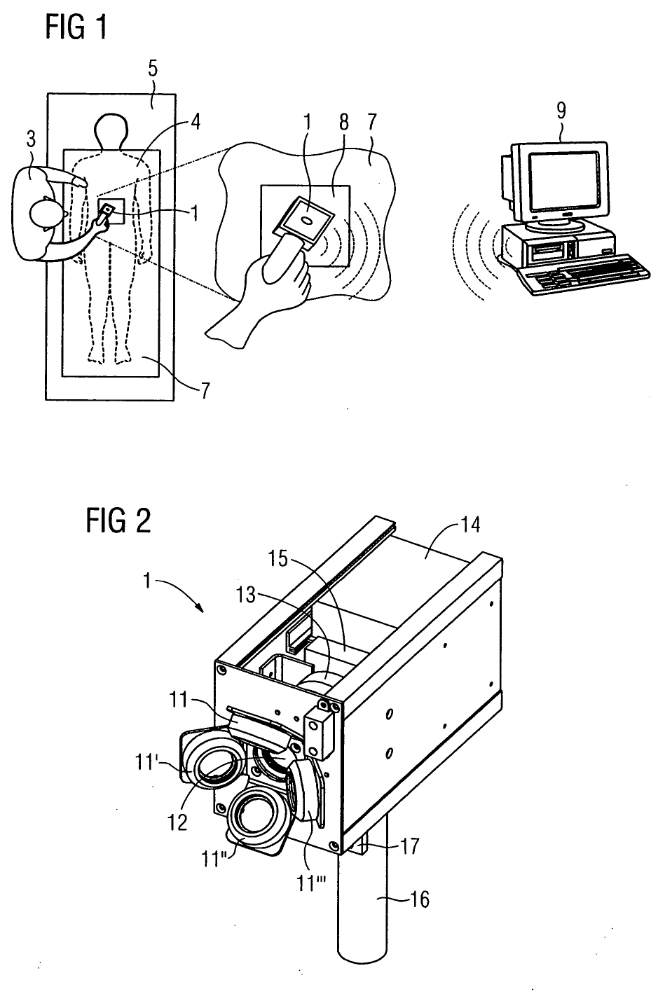

FIG. 1 schematically illustrates a scenario for using a fluorescence scanner 1. A body 4 to be examined, which may be covered by an operating room (OR) drape 7, is lying on an operating table 5. A surgeon 3 is treating a region of the body 4 through an opening in the OR drape 7. The surgeon 3 holds a fluorescence scanner 1 in his hand and with it can examine the body region to be treated.

The region 8 to be examined of the body 4 is shown schematically and enlarged. The body 4 may be covered, by the OR drape 7, except for an opening in the OR drape 7. The surgeon 3 aims the fluorescence scanner 1 at the body region 8, which can be seen and reached through the opening.

Data detected by the fluorescence scanner 1 are transmitted in cordless fashion, to a personal computer (PC) workstation 9, or the like. The PC workstation 9 displays the data received, which are image data of the body region 8 to be examined, on a screen. The surgeon 3 can view the fluorescence scan on the screen of the PC workstation 9 or other display, and thus has the outcome of the scan immediately available for viewing. The surgeon can orient the surgical strategy or planning using the fluorescence scan as needed.

To enable orientation to the image shown, the optical view of the fluorescence scan has a view of the same visible region or the same body region 8 superimposed thereon, in the form of a normal image obtained in the visible wavelength range. Based on the image in the visible wavelength range, the physician can recognize details of the body region 8 on the screen, and from the superimposed fluorescence scan, can associate the features shown on the scan with the visible points in the body region 8. Superimposition of an image made in the visible wavelength range permits the association with physical features, even if the fluorescence is in a non-visible wavelength range, such as IR.

In FIG. 2, a fluorescence scanner 1 is shown in a perspective view. The upper covering of the housing has been omitted. The fluorescence scanner 1 has a handle 16 so that it can be manipulated by the surgeon. On the handle 16, there is a button 17, with which the physician can manually initiate a fluorescence scan.

In the front region, excitation light sources 11, 11′, 11″, 11″′ are arranged such that they can illuminate a region at a distance of approximately 6 to 10 cm. For that purpose, they are arranged at an angle of approximately 45° to the front panel. This arrangement may correspond to an optimal working distance, where the scanning region is not touched by the scanner, and yet excessively high excitation light intensity may be avoided.

The excitation light sources 11, 11′, 11″, 11″′ may be based on halogen light sources To achieve fast switching times, LEDs (light emitting diodes) or laser diodes may be used, depending on the wavelengths and intensities needed. Since an individual LED has a relatively low luminous intensity, LED arrays may be used for each light source. Each of the LED arrays may have a total luminous power of approximately 0.25 to 1 Watt.

A lens 12 is aimed frontally at the illuminated region, and by means of this lens, not only fluorescent light, but ambient light may reach the fluorescence scanner 1. So that the fluorescent light will not be washed out by the ambient light, the incoming light first passes through a filter in the filter changer 13. To make a fluorescence scan, the filter allows light to pass through only in the wavelength range of fluorescence. To take a picture in the visible wavelength range, the filter changer changes to a filter that allows light in the visible wavelength range to pass through. Depending on the optical properties of the overall construction, the filter for making images based on visible light can be eliminated, and the filter changer need merely remove the fluorescence wavelength pass filter from the beam path. A fold-down mechanism of the kind known from single lens reflex cameras can be used.

Light that has passed through the filter changer 13 reaches a CCD camera 15. The CCD camera 15 is capable of recording images both in the wavelength range of visible light and in the wavelength range of the fluorescence. The image data recorded by the CCD camera 15 are received by a data acquisition unit 14 and transmitted to the outside, preferably in cordless fashion.

In an example, the fluorescence scanner I is initially operated in standard fashion, such that images are made in the visible wavelength range; that is, there is either no filter in the filter changer 13, or a filter that allows visible light to pass through, is located in the beam path. After the surgeon 3 has viewed the body region 8 in question, based on the optical image made in the visible wavelength range, a fluorescence scan is initiated. This action causes the image in the visible wavelength range to be stored in memory, and the filter changer 13 changes to a filter that allows only light in the fluorescent wavelength range to pass through. Excitation light sources 11, 11′, 11″, 11″′ are activated, and a fluorescence scan is stored in memory. From this sequence, at least if it is done fast enough, the storage in memory of one optical and one fluorescence image can be achieved from virtually the same viewing angle and the images can then be superimposed on one another.

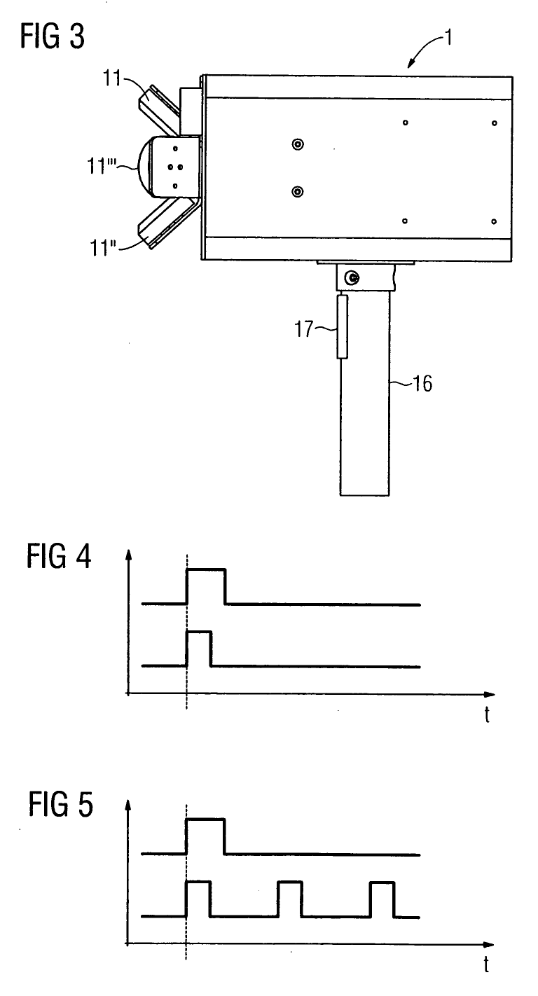

In FIG. 3, the fluorescence scanner 1 is shown in a side view. The handle 16 with the button 17 are shown, as are the excitation light sources 11, 11′, 11″, located on the front of the housing. The excitation light sources make angles of approximately 45°±20° with respect to the housing.

FIG. 4 illustrates how the excitation light sources 11, 11′, 11″, 11″′ can be pulsed. The upper curve plots the status of the button 17 over time. At the instant indicated by a dashed line, the button 17 is actuated by the surgeon in order to trip a fluorescence scan. By the actuation of the button 17, the excitation light sources 11, 11′, 11″, 11″′ are activated for a period of about 300 ms or less. The duration of the pulses is selected to be long enough to enable detecting fluorescence adequately for generating the fluorescence scan. On the other hand, the duration is short enough to avoid artifacts caused by motion (“blurring”). The pulse duration is also short enough to avoid excessive heating up of the excitation light sources 11, 11′, 11″, 11″′, and minimize the energy consumption of these light sources. The fluorescence scanner 1 has an energy source, not further shown. The energy source may be disposable batteries or rechargeable batteries, which can be accommodated, for example, in the handle 16. An integrated energy source makes a cable-bound energy supply unnecessary and makes portable operation of the fluorescence scanner 1 possible. Cable-bound energy supply may be used.

In FIG. 5, a further possible mode of operation is shown. The upper curve shows the status of the button 17 over time. At the instant indicated by a dashed line, the button 17 is actuated. The lower curve shows the state of operation of the excitation light sources 11, 11′, 11″. Tripped by the actuation of the button 17, an operating pulse with a width of approximately 300 ms or less is generated, followed by a resting phase, followed by a further operating pulse, followed by a resting phase, and so forth. The mode of operation shown in FIG. 5 makes automatically recording a succession of fluorescence scans possible.

Although only a few exemplary embodiments have been described in detail above, those skilled in the art will readily appreciate that many modifications are possible in the exemplary embodiments without materially departing from the novel teachings and advantages of the invention. Accordingly, all such modifications are intended to be included within the scope of this invention as defined in the following claims.

1. An imaging device, comprising:
an excitation light source;
a fluorescence detector; and
a data acquisition unit connected to the fluorescence detector,
wherein the excitation light source is operable in pulsed fashion.

2. The imaging device of claim 1, wherein the fluorescence detector has a filter operable to attenuate visible light.

3. The imaging device of claim 2, wherein the filter is disposed outside the optical path between a fluorescent material and the fluorescence detector and inserted in the optical path during operation of the pulsed excitation light source.

4. The imaging device of claim 1, further comprising a trigger, the excitation light source being pulsed by actuation of the trigger.

5. The imaging device of claim 4, wherein actuating the trigger initiates one light pulse from the excitation light source.

6. The imaging device of claim 1, wherein the duration of an operating pulse of the excitation light source is approximately 300 ms or less.

7. The imaging device of claim 1, wherein the excitation light source comprises a light emitting diode (LED), a laser diode, a halogen light bulb or combinations thereof.

8. The imaging device of claim 1, wherein the excitation light source is disposed to emit excitation light at an angle of between approximately 30° and approximately 45° to an optical primary axis of the fluorescence detector.

9. The imaging device of claim 1, wherein the excitation light source is operable to generate optical energy in a wavelength range of between approximately 700 nm and approximately 800 nm.

10. The imaging of claim 1, wherein the fluorescence detector is operable to detect fluorescence at wavelengths longer than approximately 700 nm.

11. The imaging device of claim 10, wherein the fluorescence detector is operable to detect fluorescence at a wavelength of approximately 780 nm

12. The imaging device of claim 1, wherein the fluorescence detector and the light source are connected to a battery contained in, or attached to, the imaging device.

13. The imaging device of claim 12, wherein image data obtained by the fluorescence detector is transmitted by modulation of data on a carrier wave, using a carrier wave in the radio frequency or optical frequency range.
